(19) United States
(12) Patent Application Publication
(10) Pub. No.: US 2010/0137706 A1
(43) Pub. Date: Jun. 3, 2010

(54) METHOD OF, AND APPARATUS FOR, CONTROLLING MEDICAL NAVIGATION SYSTEMS

(76) Inventor: Raju R. Viswanathan, St. Louis, MO (US)

Correspondence Address: Bryan K. Wheelock, 7700 Bonhomme, Suite 400, St. Louis, MO, 63105, US

(21) Appl. No.: 12/490,020

(22) Filed: Jun. 23, 2009

Related U.S. Application Data
(63) Continuation of application No. 11/184,557, filed on Jul. 19, 2005, now abandoned.
(60) Provisional application No. 60/589,273, filed on Jul. 19, 2004.

(51) Int. Cl. A61B 5/05

(52) U.S. Cl. 600/424; 600/427

(57) ABSTRACT

A method of operating a remote navigation system to that orients a medical device in a selected direction includes operating the remote navigation system to orient the medical device toward a point identified by the user on a two-dimensional map of a three-dimensional surface adjacent the medical device.

### CROSS-REFERENCE TO RELATED APPLICATIONS

This application claims the benefit of U.S. Provisional Patent Application Ser. No. 60/589,273, filed Jul. 19, 2004. The disclosure of the above-referenced application is incorporated herein by reference.

### BACKGROUND OF THE INVENTION

The present invention relates to the control of medical navigation systems, and in particular to the use of a projection map, and more specifically to the use of a conformal map in the control of medical navigation systems.

Advances in technology have resulted in systems that allow a physician or other medical professional to remotely control the orientation of the distal end of a medical device. It is now fairly routine to steer the distal end of a medical device inside a subject's body by mechanically manipulating controls on the proximal end of the medical device. Recently magnetic navigation systems have been developed that allow a physician to orient the distal end of a medical device using the field of an external source magnet. Other systems have been developed for the automated remote orientation of the distal end of a medical device, for example by operating mechanical or magnetostrictive or electrostrictive elements incorporated into the medical device. However the medical device is controlled, it can still be difficult for a physician to visualize the procedure site (which is out of view inside the subject's body), to select the desired direction in which to orient the distal end of the medical device, and to communicate the selected direction to the system in order to orient the distal end of the medical device in the selected direction.

As stated above, magnetic navigation systems have been developed which apply a controlled magnetic field to an operating region in a subject, to orient a magnetically responsive element on a medical device in the operating region. Examples of such systems include Ritter et al., U.S. Pat. No. 6,241,671, issued Jun. 5, 2001, for Open Field System For Magnetic Surgery (incorporated herein by reference). Magnetic navigation systems permit faster and easier navigation, and allow the devices to be made thinner and more flexible than conventional mechanically navigated devices which must contain pull wires and other components for steering the device. Because of the advances made in magnetic surgery systems and magnetically responsive medical devices, the determination of the appropriate field direction, and instructing the magnetic surgery system to apply the determined magnetic field are among the most difficult tasks remaining in magnetically assisted medical procedures. Significant efforts have been made to help the user to visualize the procedure, and improve the user's ability to control the magnetic surgery system during the procedure. There is often a lag between the direction of the applied field, and the actual direction of the distal end of the medical device. In some current systems, the user specifies a field direction, and mentally must take into account the lag between the applied field and the actual device direction.

For example, in the process of navigating within the heart chambers, it would be useful to have a view of the interior surface of the heart. In particular, a view such as looking up from the tricuspid or mitral valves into (respectively) right or left atrial chambers would offer a perspective directly useful for diagnostic and therapeutic purposes such as electrical activity mapping and cardiac ablation, both of which are based on access to the interior surface of the heart. An interior view is most useful when it encompasses the entire desired region in a single view, in contrast to standard “endoscopic” views which offer only a narrow field of vision.

### SUMMARY OF THE INVENTION

This invention provides a method and apparatus for controlling a medical device in a subject's body which employs a two-dimensional map of the curved surface adjacent the medical device to facilitate user operation of a remote navigation system.

### BRIEF DESCRIPTION OF THE DRAWINGS

FIG. 1 is a perspective view of a magnetic navigation system with which the method of, and apparatus for, controlling medical navigation systems of the present invention can be used;

FIG. 2 is a sample display from a navigation system showing a conformal map for controlling the navigation system in accordance with a preferred embodiment of this invention;

FIG. 3A is a sample display from a navigation system showing a conformal map for controlling the navigation system in accordance with a preferred embodiment of this invention, with a background grid;

FIG. 3B is a sample display from a navigation system showing a conformal map for controlling the navigation system in accordance with a preferred embodiment of this invention, with color coded latitude lines;

FIG. 4A is a sample display from a navigation system showing a conformal map for controlling the navigation system in accordance with a preferred embodiment of this invention, with color coordinated longitude lines;

FIG. 4B is a sample display from a navigation system showing a conformal map for controlling the navigation system in accordance with a preferred embodiment of this invention, with color coordinated longitude lines and an options window;

FIG. 5 is a sample display from a navigation system showing a conformal map for controlling the navigation system in accordance with a preferred embodiment of this invention, with color coordinated direction grid;

FIG. 6 is schematic diagram illustrating a process for creating a map of the interior of a curved surface in accordance with one embodiment of this invention;

FIG. 7 is a schematic diagram further illustrating the process for creating a map of the interior of the curved surface in accordance with one embodiment of this invention;

FIG. 8 is a schematic diagram of a map prepared in accordance with the embodiment illustrated in FIGS. 6 and 7 and described herein;

FIG. 9 is a schematic diagram of a resealed map prepared in accordance with the embodiment illustrated in FIGS. 6 and 7 and described hereon,

Corresponding reference numerals indicate corresponding parts throughout the several views of the drawings.

### DETAILED DESCRIPTION OF THE INVENTION

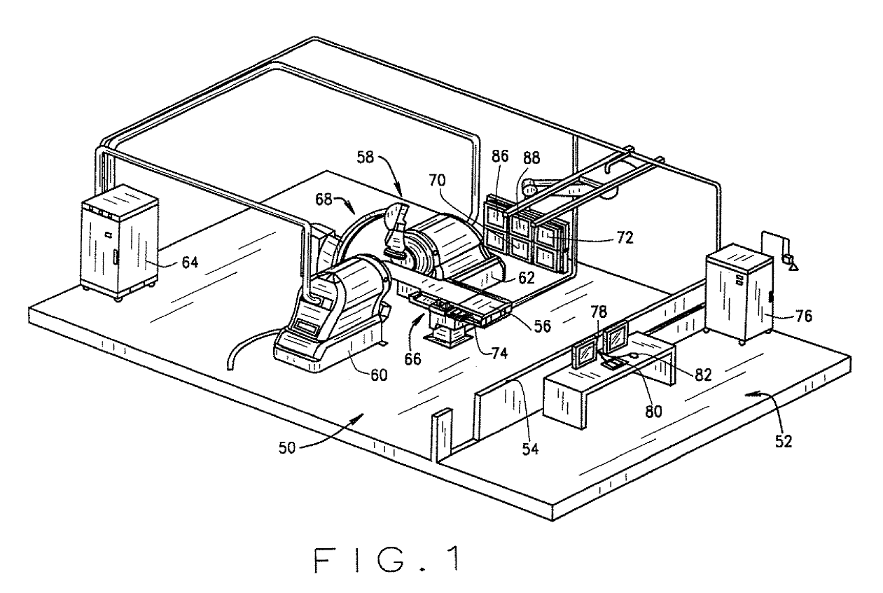

The interface methods and apparatus of the present invention can be used with any type of remotely controllable medical navigation system including, for example, mechanically, electrically, hydraulically, pneumatically, and magnetically actuable navigation systems. One possible application of the invention is in the control of magnetic navigation systems such as the magnetic navigation system shown in FIG. 1. While described primarily in connection with a magnetic surgery system, this invention is not so limited.

The interface provides a two-dimensional map of the surface of an anatomical volume or a two dimensional map of the corresponding field directions. The user can use this map (for example by moving a cursor and clicking) to identify a location or a direction on the two-dimensional anatomical map (which the interface then translates into an action by the navigation system to cause a remotely actuated medical device to move to the location or align with the direction. The user can alternatively use this map to directly identify a field direction on the two-dimensional field direction map, so that a magnetic navigation system can apply the selected magnetic field direction to the operating region.

As shown in FIG. 1, a magnetic surgery system is set up in the procedure room 50 where the subject is located, and in a control room 52. The control room 52 is preferably adjacent the procedure room 50, and there may be a window 54 between the control room and the procedure room to permit direct observation of the subject, however the control room could be remote from the subject, and with the aid of the present interface, a physician could conduct a procedure on a subject in the procedure from a control room on a different floor, in a different building, or even in a different city.

The magnetic surgery system comprises a subject bed 56, and a magnetic navigation system 58 comprising opposed magnet units 60 and 62 on opposite sides of the bed operated by a processor 64 and controlled by controls 66 adjacent the bed 56. An imaging system 68, such as an x-ray imaging system on a C-arm, displays images of the operating region on a monitor 70 in the procedure room 50. The interface system of the present invention provides a convenient way for a user to operate the magnetic navigation system 58 to control the distal end of a medical device in the operating region inside the subject's body.

The interface includes a display on, for example, an LCD monitor 72, and a digital tablet 74 in the procedure room 50, a processor 76, a display on, for example, monitor 78, a key board 80, and a mouse/digital tablet 82 in the control room 54. Additional displays on monitors 86 and 88 can be provided in the procedure room 50 which integrate images from the imaging system 68 with the interface. One or more additional monitors 90 can be provided in the control room so that the images are available in the control room as well. The monitor 90 preferably displays a multi-pane display.

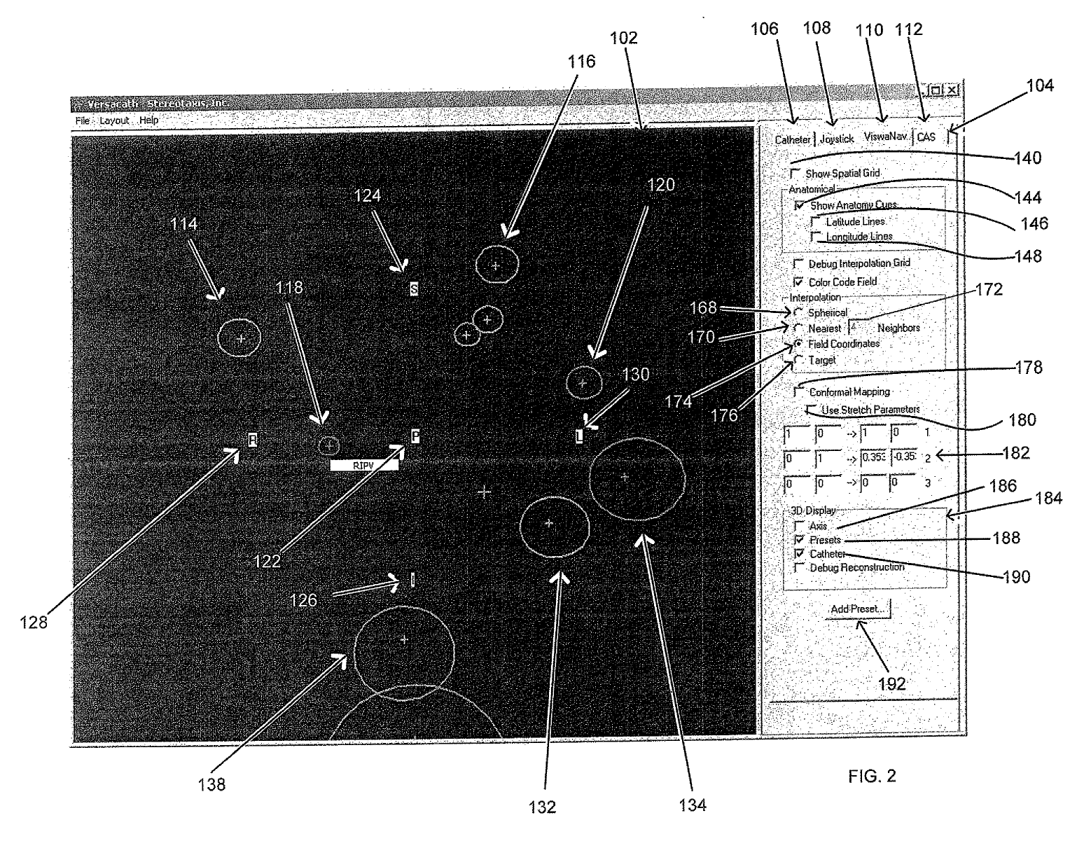

A sample display from a navigation system employing a conformal map display in accordance with the principles of this invention is indicated generally as 100 in FIG. 2. The display 100 comprises a display pane 102 for displaying the conformal map, and a control pane 104 for controlling the display of the conformal map on the display pane 102. Using tabs 106, 108, 110, and 112, the user can select one of several modes of operation of the navigation system, for example using tab 110 to enter the conformal map mode of navigation to use a conformal map to identify locations or directions to the navigation system. As shown in FIGS. 2-5, the conformal map can be created using a standardized or idealized anatomical structure, and preferably registered with the subject's anatomy. Alternatively the conformal map can be made from the subject's anatomy, for example from preoperative or intraoperative CT or MR imaging.

The conformal maps illustrated in FIGS. 2-5 are of the left atrium of a human heart, but the invention is not so limited and this invention can be applied to other chambers of the heart, or other anatomical spaces. The resulting anatomical conformal map provides a convenient way of identifying locations and directions in a three-dimensional space, utilizing a two-dimensional screen. When used with a magnetic navigation system, a field conformal map provides a convenient way of directly identify a desired magnetic field for the magnetic navigation system to apply to the operating region.

Generally, the invention provides a method of, and embodiments of apparatus for, controlling a remote medical navigation system to orient a medical device in an operating region in a subject's body. This invention can be employed with any remote navigation system capable of orienting a medical device in a selected orientation, as well as any remote navigation system capable of orienting and advancing a medical device in a selected direction. This specifically includes magnetic navigation systems that use an externally applied magnetic field to orient a device with a permanent or variable magnetic moment; as well as devices with mechanical, pneumatic, hydraulic, electrostrictive, and magnetostrictive navigation systems that orient a medical device in a selected orientation.

In the preferred embodiment, three-dimensional image data of the operating region in the subject is obtained, for example from MR or CT imaging, or from any other suitable source of three dimensional image data. As exemplified in this preferred embodiment, the operating region might include the left atrium of the subject's heart, however, the invention is not so limited, and the operating region can include any portion of a subject's body in which there is sufficient space to navigate a medical device. Image data of the operating region, in this preferred embodiment an MR image of the subject's left atrium, is obtained and processed. The pulmonary veins are truncated, leaving generally circular openings in wall of the left atrium.

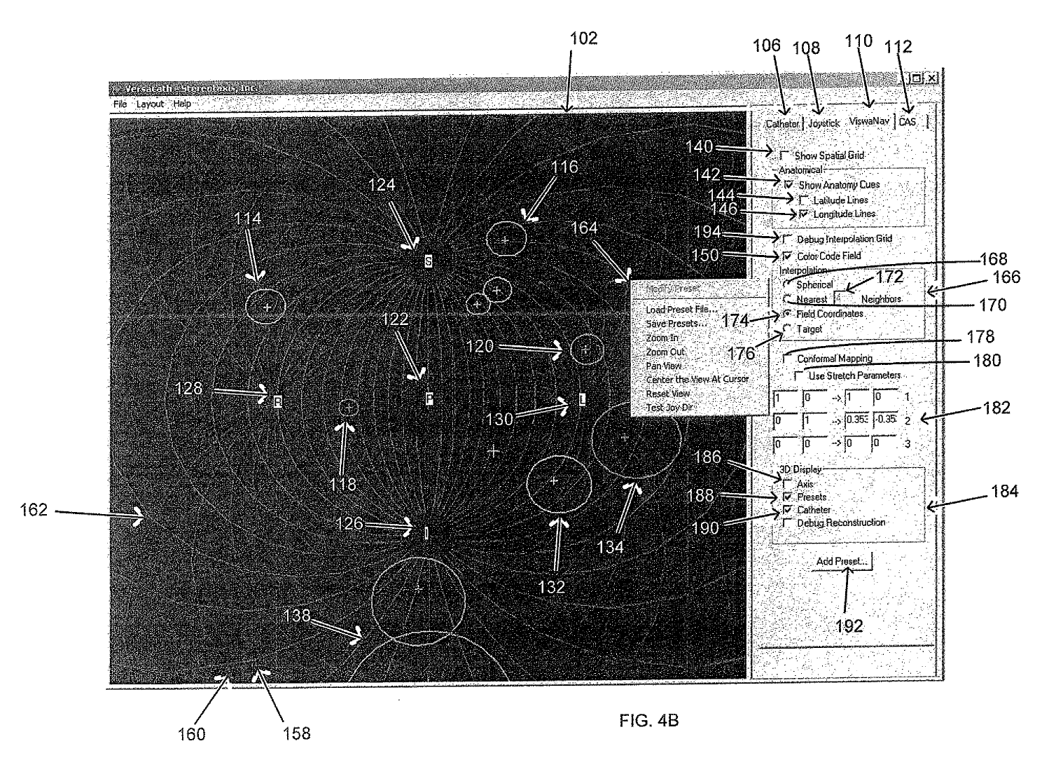

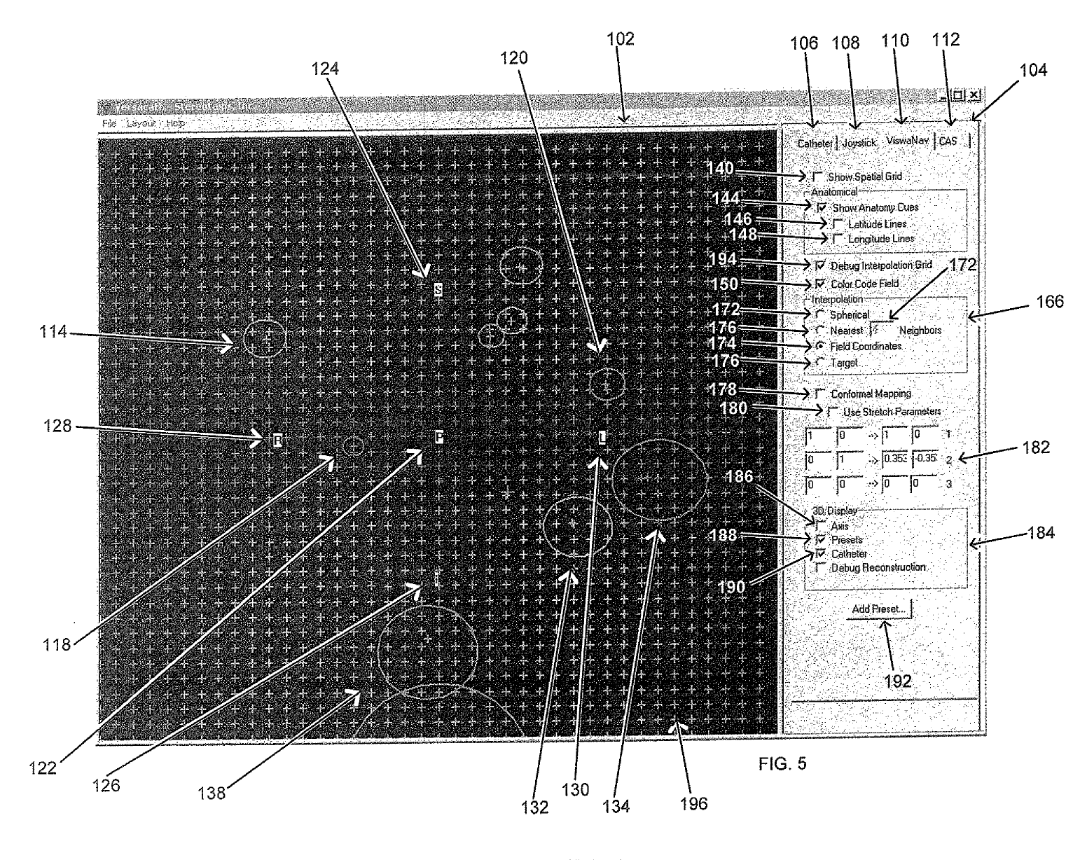

As illustrated in FIGS. 2-5, and explained in more detail below with reference to FIGS. 6 through 9, the volume is cut along a first plane generally opposing the surface of interest, forming a generally circular boundary for the map. A mapping point is selected proximal to the boundary, and each point on the surface of the volume and more particularly of the three dimensional image of the volume is projected to a second plane spaced distally behind the three dimensional image. The first and second planes are preferably generally parallel. This results in the formation of a two dimensional nearly conformal map of at least a portion of the surface of the volume (in this preferred embodiment the left atrium of the subject's heart). Although the description discusses conformal maps, other projections that result in minimal distortions could be implemented in accordance with the principles of this invention.

It is a feature of conformal maps that angular relationships and shapes are generally preserved. Thus in FIGS. 2-5, the four pulmonary veins appear as generally circular openings. The right superior pulmonary vein is indicated as 114, the left superior pulmonary vein is indicated as 116, the right inferior pulmonary vein indicated as 118, and the left inferior pulmonary vein indicated as 120 on the two-dimensional conformal map on the display pane 102. Each point on the two dimensional conformal map corresponds to a point on the preoperative or intraoperative image of the operating region, which in turn corresponds to a point on the three dimensional surface of the subject's left atrium. By registering the image with the navigation system, picking a point on the two dimensional conformal map generated from the image corresponds to picking a point on the three dimensional image, which identifies that point to the remote navigation system. Similarly, when using a standardized or idealized anatomical model, by registering landmarks on the anatomical model with landmarks on the subject anatomy in the navigation system frame of reference, picking a point on the two dimensional conformal map generated from the image corresponds to picking a point on the three dimensional image, which corresponds to identifies that point to the remove navigation system.

Because of the properties of a conformal map, a circle around one of the pulmonary vein openings 114, 116, 118 or 120 on the conformal map 112 corresponds to a circle around the corresponding pulmonary vein opening in the subject's left atrium, and a direct, minimal length line between two pulmonary vein openings on the conformal map 112 corresponds to a minimal length line between the corresponding pulmonary vein openings in the subject's left atrium.

In addition to facilitate the selection and identification of points to the navigation system, other directional landmarks can be displayed, for example markers of anatomical direction can be displayed, including for example a marker “P” indicated as 122 can indicate the posterior direction, a marker “S” indicated as 124 can indicate the superior direction, a marker “I” indicated as 126 can indicate the inferior direction; a marker “R” indicated as 128 can identify the right lateral direction, and a marker “L” indicated as 130 can identify the left lateral direction. Other anatomical features marked on the image can also be mapped onto the conformal map. For example positions around a structure, such as the mitral valve can also be identified on the conformal map. Thus, as shown in FIG. 2, the 12 o′clock position on the mitral valve can be identified by marker 132, the three o′clock position on the mitral can be identified with marker 134, and the nine o′clock position on the mitral valve can be identified with marker 138. Of course other anatomic indicators can be provided to indicate to the user locations and directions on the conformal map that correspond to desired locations and direction in the operating region.

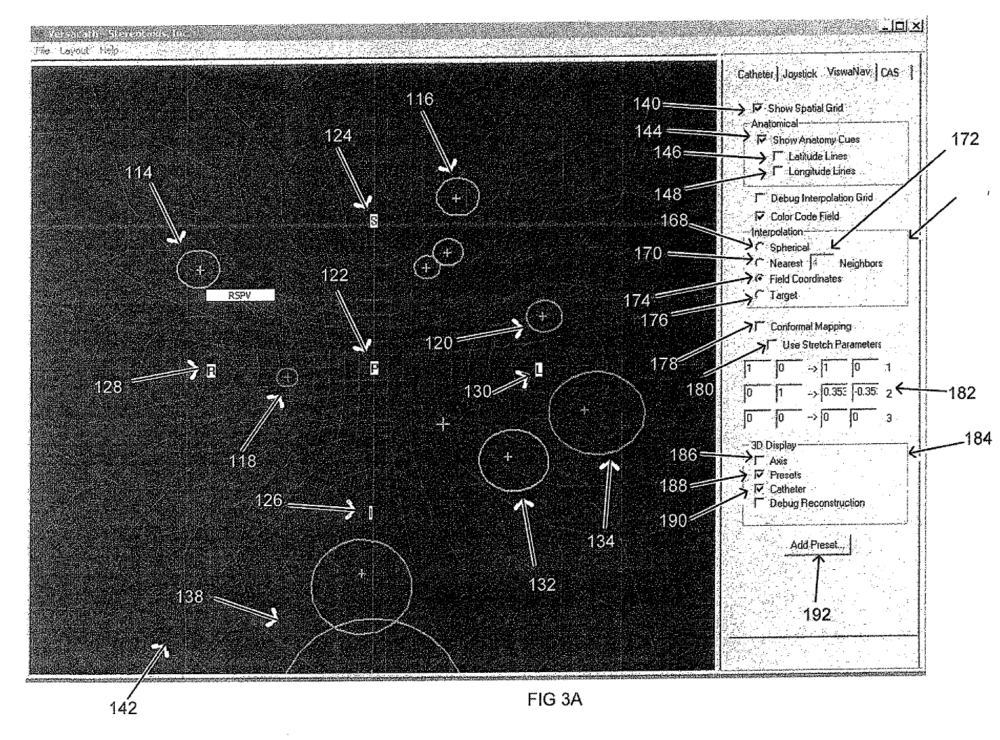

As shown in FIG. 3A, the control pane 104 can have a select box 140 which the user can operate (for example by pointing a cursor with a mouse or joystick, and clicking a control button), to display a grid 142 (shown in blue in FIG. 3A), to facilitate correlating locations and direction on the conformal map. As shown in FIG. 3B, the control pane 104 can have a select box 144 which the user can operate (for example by pointing a cursor with a mouse or joystick, and clicking a control button), to display preselected anatomical markers 122-130 described above. The control pane preferably also has select boxes 146 and 148 which the user can operate (for example by pointing a cursor with a mouse or joystick, and clicking a control button), to display lines of latitude and/or lines of longitude relative to the anatomical directions, respectively. The control pane preferably also has select box 150 which the user can operate (for example by pointing a cursor with a mouse or joystick, and clicking a control button), to color coordinate the marks associated with the anatomical directions (that are enabled with select box 144). Thus, as shown in FIG. 3B, the box 144 is selected to display the lines of latitude around each of the anatomical directions, and thus the pane 102 has blue lines of latitude 152, corresponding to the blue color of the posterior indicator 122, green lines of latitude 154, corresponding to the green color of the superior and inferior indictors 124 and 126, and red lines of latitude 156 corresponding to the red color of the right and left lateral indicators 128 and 130. Of course some other color scheme could be used, but it is desirable that the colors of the latitude lines be logically associated with the colors of the direction indicators.

As shown in FIG. 3B, the anatomical markers box 144 is also checked, as is the color code box 150, so the anatomical markers 122-130 are displayed on the screen in color (rather than in monotone, as they would appear if the box 150 were not checked). The color of each of the anatomical markers 122-130 is coordinated with the anatomical direction with which it is most nearly associated. Thus, markers 116 and 138 are most closely associated with the superior and inferior directions, respectively, and are therefore colored green to coordinate with the latitude lines 154. The markers 118 and 134 are most closely associated with the right lateral, and left lateral directions, respectively, and are therefore colored red to coordinate with the latitude lines 156. The other markers, 114, 132 and 120, have colors other than the blue, green, and red to indicate that they are between two directions.

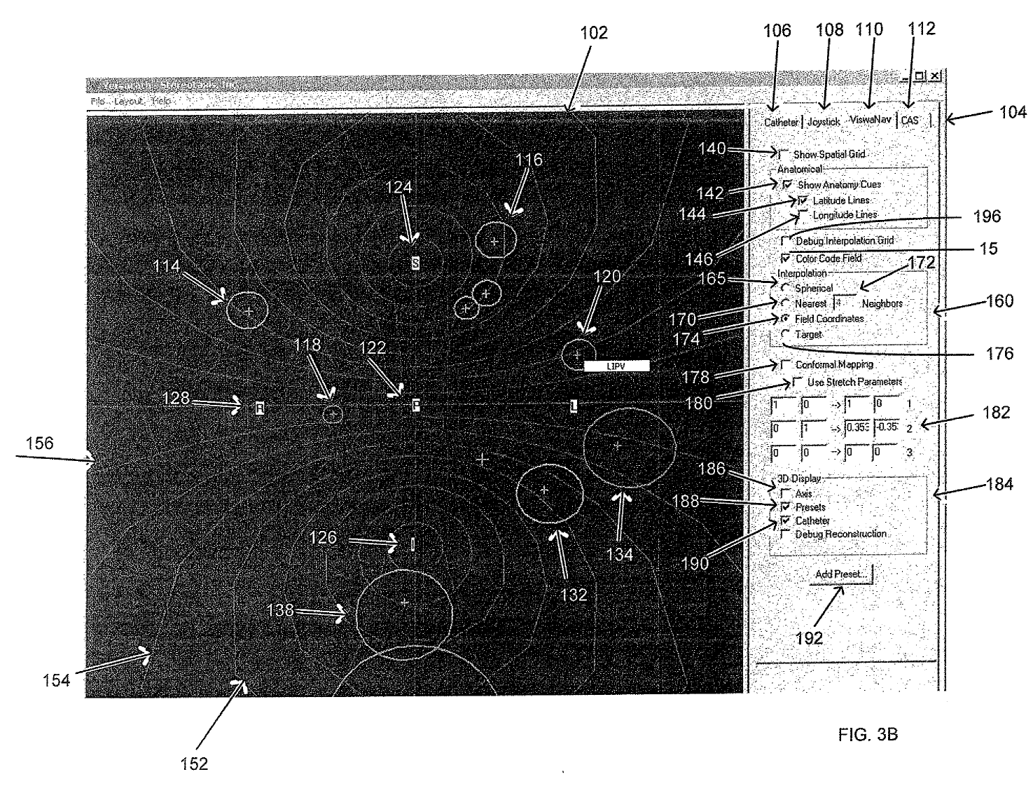

As shown in FIGS. 4A and 4B, the box 146 is selected to display lines of longitude around each of the anatomical directions, and the pane 102 has blue lines of longitude 158, corresponding to the blue color of the posterior indicator 122, green lines of longitude 160, corresponding to the green color of the superior and inferior indictors 124 and 126, and red lines of longitude 160 corresponding to the red color of the right and left lateral indicators 128 and 130. Of course some other color scheme could be used, but it is desirable that the colors of the latitude lines be logically associated with the colors of the direction indicators.

As shown in FIGS. 4A and 4B, the anatomical markers box 144 is also checked, as is the color code box 150, so the anatomical markers 122-130 are displayed on the screen in color (rather than in monotone, as they would appear if the box 150 were not checked). The color of each of the anatomical markers 122-130 is preferably as described above with respect to FIG. 3B, although some other color scheme could be used.

As shown in FIG. 4B, right clicking on the display pane 102 causes a box 164 to pop up, from which the user can select functions such as “Load Presets”, “Save Presets”, “Zoom In”, “Zoom Out”, “Pan View”, “Center the View at Cursor”, “Reset View”, and “Test Joy Controller”. The “Load Presets” allows the user to load a set of predetermined locations or directions into the navigation system, so that these preset locations or directions can be displayed on the conformal map on the display pane 102. These presets can be pre-stored in the system, or created by the user.

The “Zoom In” function allows the user to zoom in on the conformal map on the display pane 102. The “Zoom Out” function allows the user to zoom out on the conformal map on the display pane 102. The “Pan View” function allows the user to pan across the conformal map on the display pane 102. The “Center the View at Cursor” function puts the point at the cursor at the center of the display pane 102. The “Reset View” function allows the user to reset the position of the conformal map on the display pane 102 to a default position. The “Test Joy Controller” enables a joy stick connected to the system to control the cursor on the display.

The control pane 104 preferably also has a Interpolation box 166, with a Spherical pick button 168. A Nearest pick box 170, with an associated numerical indicator box 172, a Field Coordinates button 174, and a Target button 176. In the preferred embodiment, the navigation system is a magnetic navigation system, and the Field Coordinates button 174 and the Target button 176 allows the user to toggle between the Field Coordinates mode, in which the conformal map in pane 102 displays magnetic field directions, and a Target mode, in which the conformal map on pane 102 displays locations. Because of the physical properties of the medical device being navigated, there is a lag between the magnetic field direction applied to a magnetically responsive medical device and the actual direction of the magnetic medical device. In the Field Coordinates mode the map displays and allows the user to directly select a field direction corresponding to the various anatomical and other features displayed on the map. In the Target mode the map displays and allows the user to select a location or direction, and the interface determines the correct field direction for the magnetic navigation system to apply to reach the selected location or direction.

The Spherical pick button 168 and the Nearest pick box 170 allows the user to select the method of interpolation when a point is selected between the preset field directions in the Field Coordinates mode. In the Spherical interpolation mode, when the user selects a point on the conformal map between known directions, all of the known directions are used in an interpolation to determine the direction corresponding to the point selected on the conformal map. In the Nearest interpolation mode, when the user selects a point on the conformal map between known directions, a selected number of nearest known directions (selected in box 174) are used in an interpolation to determine the direction corresponding to the point selected on the conformal map.

The control pane 104 preferably also has a Conformal Mapping box 178, and a Use Stretch Parameters box 180, and associated numerical indicator boxes 180. These boxes 180 allow the user to select scaling values scaling the conformal map. Alternatively, these values can be preset to optimum levels so the user merely has to select whether or not to scale the conformal map in box 178.

The control pane 104 preferably also has a 3D Display box 184 that allows the user to select features from a 3-dimensional display to display on the pane 102. The control pane 104 also has an axis select 186 box, which allows the user to select whether or not to display the major anatomical axis (when the system is in the Target mode). The control pane 104 also has a Presents Select box 188 that allows the user to select whether or not to display certain preset directions (when the system is in the Target Mode). The control panel 104 also has a Catheter select box 190 that allows the user to select whether or not to display the distal end of the medical device.

The control pane 104 also has a Add Presets button 192 that allows the user to add selected directions to the preset directions available for display on the pane 104.

The resulting two dimensional conformal map of the three-dimensional interior surface of the subject's left atrium can be displayed, and a user can indicate or input a selected direction to the remote navigation system by selecting a point on the two-dimensional conformal map. Either through a look-up table or through data processing, a processor can correlate a point selected by the user on the map with a point on the three dimensional image of the subject's atrium. This unique point can then be provided to the remote navigation system, in this preferred embodiment a magnetic navigation system. The magnetic navigation system can determine the direction between the present location of the medical device and the selected point, and operate to cause the medial device to point to the selected point by applying an appropriate magnetic field. (Of course, with some other type of navigation system, the system would operate to orient the medical device in the selected direction.)

There are a variety of ways for a user to select a point. The image could be displayed on a pressure sensitive display, so that the point selected by the user can be indicated with a stylus other similar device. Alternatively, a cursor can be provided, under the control of a device such as a mouse or joystick for the user to manipulate the cursor and select a point.

In this preferred embodiment, the openings 114, 116, 118 and 120 for the pulmonary veins provide landmarks for orienting the user. In addition, and in other operating regions in the body without convenient anatomical markers, various frames of reference can be superposed on the conformal map. For example, portions corresponding to octants of a sphere can be indicated by color coding or otherwise. Furthermore, the actual points marked by the user can continue to be displayed, providing additional points of reference to the user.

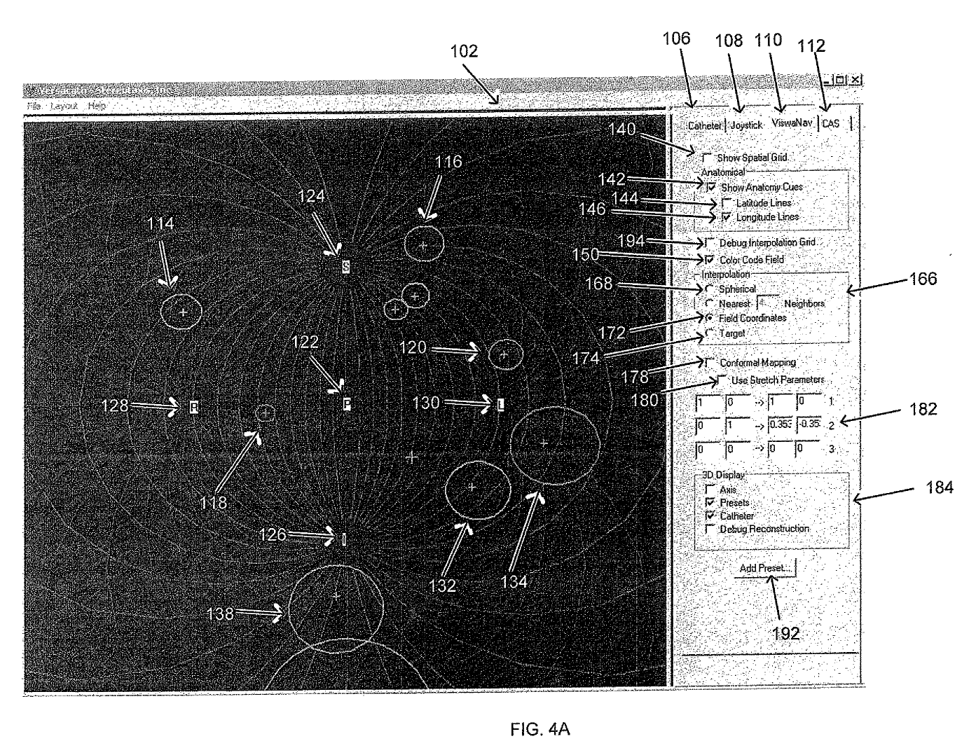

An alternative display of the conformal map is shown in FIG. 5. The control pane 104 preferably includes a Interpolation Grid box 194, which when actuated displays a grid of color coded markers 196 indicating a magnetic field direction For example, as shown in FIG. 5, the markers 196 in the vicinity of the posterior indicator 122 are colored blue corresponding to the color of the posterior indicator, the markers 196 in the vicinity of the superior and inferior indicators 124 and 126 are colored green, corresponding to the color of the superior and inferior indicators, and the markers 196 in the vicinity of the right lateral and left indicators 128 and 130 are colored red, corresponding to the colors of the right lateral and left lateral indicators. Indicators corresponding to directions between the direction indicators 122-130 have intermediate colors.

More specifically, one possible method for creating a map of the interior curved surface 200 as seen from an opening 202 is illustrated in FIG. 6. The normal 204 to the plane 206 of the opening 202 is the vertical axis in FIG. 6. It is convenient to find the smallest sphere 208 enclosing the surface 200. This can be accomplished as follows: Opening 202 lies in a plane 206. For each of a grid of points Oᵢ in plane 206, a vertical line Vᵢ intersecting the surface 200 is constructed.

For every point on Vᵢ, the perpendicular distance L to surface 200 is Lᵢ, and the maximum distance is Lᵢ*. The point O* in plane 206, such that maximum distance L* is least among grid points Oᵢ is found. H is the maximum distance L* for the point O* and S is the point on line V* through point O* corresponding to L*, i.e., where L=H. Point S then, is the center of the smallest sphere enclosing the surface 200, and H is the radius of that sphere. P* is a pole of the sphere a distance H down from S.

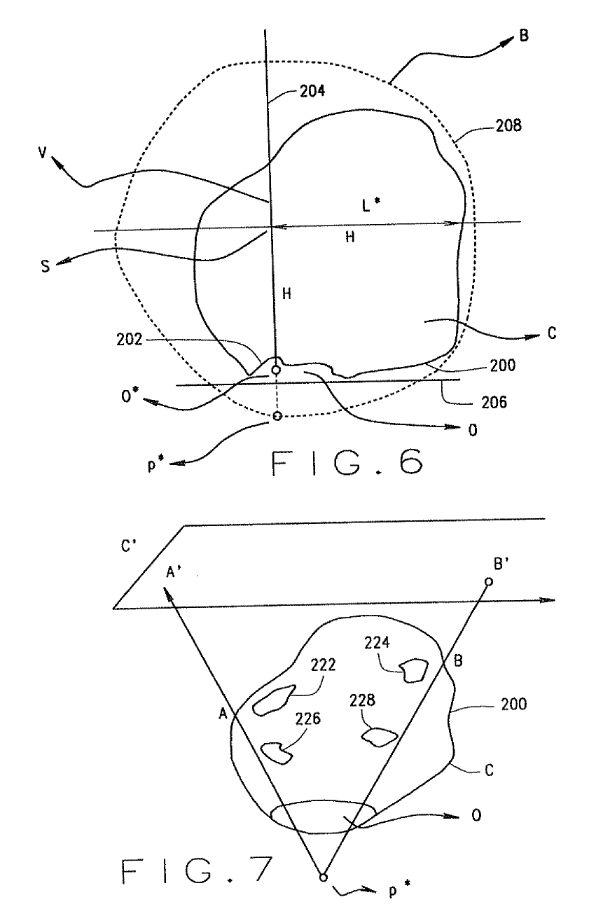

Once the pole P* is determined, the surface C may be mapped onto a horizontal plane M by a stereographic-like projection. Point A on C goes to point A¹ on M, and point B on C goes to point B¹ on M. This results in a “flat” representation C¹ of C on M, shown in FIG. 8.

Any holes (e.g., pulmonary vein ostia 222, 224, 226, and 228) in the surface 200 would also be mapped as holes 222′, 224′, 226′ and 228′ in C¹. Opening O at the base of C maps to the boundary of C¹.

In general, the size and/or aspect ratio of C¹ could be quite large. To ensure a relatively uniform scaling, a further conformal mapping can be performed. Consider a point x (=(x+iy) for a point (x, y) in C¹) written as a complex variable. A map can be written:

$$w = λ = ( \frac{a + z}{b + z} )$$

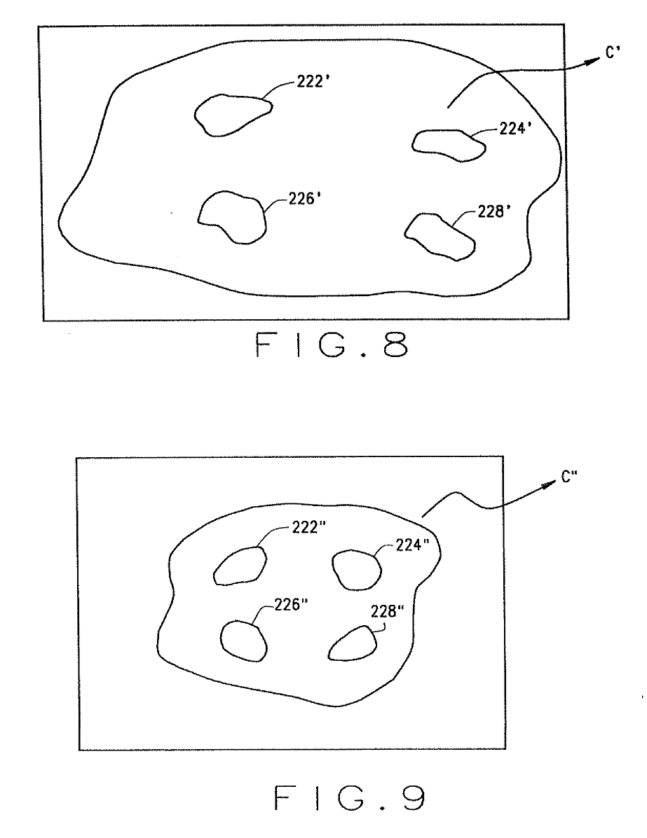

As a new representation C¹¹ of C¹, so that every point x in C¹ goes to a point w in C¹¹, illustrated schematically in FIG. 9. As shown in FIG. 9, the holes 222, 224, 226, and 228 in the surface 200, represented as holes 222′, 224′, 226′, and 228′ in map C¹ in FIG. 8, are represented as holes 222″, 224″, 226″, and 228″ in map C¹¹ in FIG. 9. The corresponding inverse map is

$$z = \frac{( λ a - bw )}{( w - λ )}$$

The parameters (λ, a, b) are determined by specifying desired mapping locations for 3 points in C¹. For example, these could be respectively the centroid, the maximum −y location, and the farthest location on a pulmonary vein (all in C¹) which map onto

$$w = ( \frac{1}{2} , \frac{1}{2} ) ,$$

respectively. Various other choices are of course possible.

In practice it may be preferable to allow the user to define one or more of these 3 known mapped points (together with mapped locations).

Once these 3 points are defined, the parameters (λ, a, b) are determined by solving a system of 3 algebraic equations. The final map C¹¹ that is obtained is a minimal distortion map in the sense that it is a near-conformal representation of the original image data/(interior) surface C. This means that angles are locally preserved, so that a line making (for instance) a 90° angle with a pulmonary vein ostium when it intersects in C¹¹ would do nearly likewise in C.

Thus a physician can define ablation paths etc. in the flat projection C¹¹, and since the inverse map is defined, the corresponding path on the endocardial surface C is defined.

The catheter tip can be made to track an appropriate path in 3D space based upon path definitions made on a mapped per-operative image (it is assumed that a suitable registration can be performed).

Thus, target navigation may be enabled on the mapped pre-operative image. Likewise, a joystick can be mapped to this mapped pre-operative image for continuous navigation.

This technique of displaying a near-conformal flat projection of a curved surface, interior or exterior, generalizes to other organs and is generally useful in medical navigation applications. A single view or display can capture the entire curved surface data set and is a distinct advantage over “endoscopic” or narrow field-of-view displays.

When registered to an x-ray system, the current device tip orientation and/or location may be shown on the mapped image as well.

What is claimed is:

1. A method of operating a remote navigation system to that orients a medical device in a selected direction, the method comprising: operating the remote navigation system to orient the medical device toward a point identified by the user on a two-dimensional map of a three-dimensional surface adjacent the medical device.

2. The method according to claim 1 wherein the two-dimensional map is a conformal map.

3. The method according to claim 1 wherein the two-dimensional map is a projection of a curved surface from a projection point proximal to the mapped three-dimensional surface to a projection plane distal to the mapped three dimensional surface.

4. The method according to claim 3 wherein the two-dimensional projection is made from a three-dimensional pre-procedure image of the operating region in the subject.

5. The method according to claim 3 wherein the two-dimensional projection is made from an idealized three dimensional image of the operating region.

6. The method according to claim 1 wherein the remote navigation system is a magnetic navigation system that applies a magnetic field to orient the medical device in the selected direction.

7. The method according to claim 1 wherein the medical device is an elongate medical device having a distal end, and wherein the remote navigation system orients at least the distal end of the device.

8. The method according to claim 7 wherein the remote navigation system is a magnetic navigation system that applies a magnetic field to orient the distal end of the medical device in the selected direction.

9. A method of operating a remote navigation system that automatically orients the distal end of an elongate medical device in a selected direction, the method comprising operating the remote navigation system to orient the medical device in a direction aligned with a point selected by the user on a three-dimensional surface adjacent the medical device by identifying the point on a two dimensional map of the surface.

10. A method of operating a navigation system that automatically orients a medical device in a selected direction, the method comprising:
accepting as an input of the selected direction, an indication of a point on a three-dimensional surface adjacent the medical device made by identifying a point on a two-dimensional map of the three dimensional surface; and
operating the navigation system to cause the medical device to orient in the selected direction.

11. A method of operating a remote navigation system that automatically orients a medical device in a selected direction, the method comprising:
accepting an input of a selected direction from a user by the user's identification of a point on a two-dimensional map of a three-dimensional surface adjacent the medical device; and
controlling the remote navigation system to apply a magnetic field to align the medical device in the direction input by the user.

12. A method of operating a magnetic navigation system that automatically orients a medical device in a selected direction, the method comprising:
automatically operating the magnetic navigation system to apply a magnetic field to orient the medical device toward a point on a surface adjacent to the medical device selected by the user on a two dimensional map of the surface.

13. A method of operating a magnetic navigation system that applies a magnetic field to a magnetically responsive medical device in a cavity in an operating region in a subject's body, the method comprising selecting a direction by indicating a point on a two-dimensional map of at least a portion of the surface of the cavity to apply a magnetic field in a direction to cause the magnetically responsive medical device to orient toward the selected point on the surface of the cavity.

14. A method of operating a magnetic navigation system that applies a magnetic field to a magnetically responsive medical device in a cavity in an operating region in a subject's body, the method comprising indicating a direction by selecting a point on the surface of the cavity by indicating a point on a two-dimensional projection of the three-dimensional surface of the cavity to apply a magnetic field in a direction to cause the magnetically responsive medical device to orient toward the selected point on the cavity.

15. The method according to claim 14 wherein the two-dimensional projection is made from a three-dimensional pre-procedure image of the operating region in the subject.

16. The method according to claim 14 wherein the two-dimensional projection is made from an idealized three dimensional image of the operating region.

17. An interface for operating a remote navigation system to that orients a medical device in a selected direction, the interface comprising a display for displaying a two-dimensional map of a three-dimensional surface adjacent the medical device; an input device for selecting a point on the two-dimensional map on the display; a processor for determining a direction corresponding to the point selected with the input device.

18. The interface according to claim 17 wherein the two-dimensional map is a conformal map.

19. The interface according to claim 17 wherein the two dimensional map is a projection of a curved surface from a point proximal to the mapped three-dimensional surface onto a plane distal of the mapped three dimensional surface.

20. The interface according to claim 17 wherein the remote navigation system is a magnetic navigation system that applies a magnetic field to orient the medical device in the determined direction.

21. The interface according to claim 17 wherein the medical device is an elongate medical device having a distal end, and wherein the remote navigation system orients at least the distal end of the device.

22. The interface according to claim 21 wherein the remote navigation system is a magnetic navigation system that applies a magnetic field to orient the medical device in the selected direction.

23. An interface for operating a remote navigation system that automatically orients the distal end of an elongate medical device in a selected direction, the interface comprising a display displaying a two-dimensional map of a surface adjacent the medical device, an input device for selecting a point on the two dimensional map to indicate a direction.

24. An interface for operating a remote navigation system that automatically orients the distal end of an elongate medical device in a selected direction, the interface comprising a display displaying a two-dimensional map of a surface adjacent the medical device, an input device for selecting a point on the two dimensional map to indicate a direction.; and a controller for operating the remote navigation system to orient the distal end of the medical device in a direction aligned with a point on the surface corresponding to the point selected on the two dimensional map.

25. An interface for operating a remote navigation system that automatically orients a medical device in a selected direction, the interface comprising:
a two-dimensional map of the three dimensional surface;
an input device for inputting a selected direction by selecting a point on the two-dimensional map; and
a controller for controlling the remote navigation system to apply a magnet field to align the medical device in the direction of the point identified by the user.

26. An interface for operating a remote navigation system that automatically orients a medical device in a selected direction, the interface comprising:
a controller for operating the navigation system to apply a magnetic field to orient the medical device toward a point on the surface of a chamber selected on a two dimensional map of the surface.

27. A remote navigation system for operating a remote navigation system to orient a medical device in a selected direction, the system comprising: a display of a two-dimensional map of a three-dimensional surface adjacent the medical device; an input device for selecting a point on the two-dimensional map; a positioning system for orienting the medical device toward a point identified with the input device on the two-dimensional map.

28. The remote navigation system according to claim 27 wherein the two-dimensional map is a conformal map.

29. The remote navigation system according to claim 27 wherein the two-dimensional map is a projection of a curved surface from a point proximal to the mapped three-dimensional surface to a plane distal to the mapped three-dimensional surface.

30. The remote navigation system according to claim 27 wherein the positioning system is a magnetic navigation system that applies a magnetic field to orient the medical device in the selected direction.

31. The remote navigation system according to claim 27 wherein the medical device is an elongate medical device having a distal end, and wherein the remote positioning system orients at least the distal end of the medical device.

32. The remote navigation system according to claim 31 wherein the remote positioning system is a magnetic navigation system that applies a magnetic field to orient the medical device in the selected direction.

33. A remote navigation system that automatically orients the distal end of an elongate medical device in a selected direction, the system comprising a display displaying a two-dimensional map of a surface adjacent to the medical device, and an input device for indicating a point on the two-dimensional display, and a controller to cause the remote navigation system to orient the medical device in a direction aligned with the point on the surface selected by the user on the two-dimensional map of the surface.

34. A remote navigation system that automatically orients a medical device in a selected direction the system comprising: a display displaying a two-dimensional map of a three-dimensional surface adjacent the medical devices; and input device for indicating a point on the two-dimensional map of the three dimensional surface; and a controller causing the magnetic navigation system to align the medical device in the direction of the point on the three dimensional surface corresponding to the indicated point on the two-dimensional map.

35. A remote navigation system that automatically orients a medical device in a selected direction, the system comprising: a display of a two-dimensional map of a surface adjacent the medical device, in input device for inputting a point on the two-dimensional map; and a magnet system that applies a magnet field to align the medical device in the direction of the point on the surface corresponding to a point input on the two-dimensional map.

36. A remote navigation system that automatically orients a medical device in a selected direction, the system comprising: a magnetic navigation system that automatically operating the navigation system to apply a magnetic field to orient the medical device toward a point on the surface of chamber selected by the user on a two dimensional map of the surface.

37. A magnetic navigation system that applies a magnetic field to a magnetically responsive medical device in a cavity in an operating region in a subject's body, the system comprising a two-dimensional projection of at least a portion of the three-dimensional surface of the cavity; a magnetic system that applies a magnetic field in a direction to cause the magnetically responsive medical device to orient toward the selected point on the three-dimensional surface corresponding to a point on the two-dimensional map selected by the user.

38. A magnetic navigation system that applies a magnetic field to a magnetically responsive medical device in a cavity in an operating region in a subject's body, the system comprising a two-dimensional projection of the three-dimensional surface of the cavity;
a magnet system applying a magnetic field in a direction to cause the magnetically responsive medical device to orient toward a point on the cavity corresponding to a point selected on the two-dimensional projection.

39. The magnetic navigation system according to claim 38 wherein the two-dimensional projection is of a three-dimensional pre-procedure image of the cavity.

40. The magnetic navigation system according to claim 39 wherein the two-dimensional projection is of an idealized three-dimensional image of the cavity.
